(19) United States
(12) Patent Application Publication
(10) Pub. No.: US 2026/0290604 A1
(43) Pub. Date: Sep. 24, 2026

(54) Peripheral artery disease step counter

(71) Applicant: BIOTRONIK SE & Co. KG, Berlin (DE)

(72) Inventor: Thomas DOERR, Berlin (DE)

(73) Assignee: BIOTRONIK SE & Co. KG, Berlin (DE)

(21) Appl. No.: 19/474,449

(22) PCT Filed: Apr. 17, 2024
(86) PCT No.: PCT/EP2024/060412

§ 371 Date: Oct. 10, 2025

(30) Foreign Application Priority Data
Apr. 21, 2023 (EP) 23169134.6

(51) Int. Cl. G16H 50/30; G16H 10/60

(52) U.S. Cl. CPC G16H 50/30; G16H 10/60

(57) ABSTRACT

A system for estimation of a peripheral artery disease (PAD) status of a patient, the system includes means for receiving motion data including at least one motion parameter and means for determining an estimate PAD status, based at least partly on the motion data.

### CROSS-REFERENCE TO RELATED APPLICATIONS

This application is the United States National Phase under 35 U.S.C. § 371 of PCT International Patent Application No. PCT/EP2024/060412, filed on Apr. 17, 2024, which claims the benefit of European Patent Application No. 23169134.6, filed on Apr. 21, 2023, the disclosures of which are hereby incorporated by reference herein in their entireties.

### TECHNICAL FIELD

The invention relates to systems, methods, and computer programs for monitoring the progress of users with peripheral arterial disease (PAD) based on the evaluation of disease-typical maximum walking distances.

### BACKGROUND

Currently, progression monitoring in PAD patients takes place cyclically through regular visits to the doctor (e.g., a general practitioner, an angiologist, etc.). This in-person monitoring is associated with considerable effort and costs. Also, deteriorations are often detected late (long intervals between visits to the doctor) and therefore a necessary therapy is initiated late.

Altogether, this renders current approaches expensive and inefficient. Therefore, there is still a need to further improve approaches for monitoring the progress of PAD patients.

The various aspects described herein solve the above problem at least in part.

The present disclosure is directed toward overcoming one or more of the above-mentioned problems, though not necessarily limited to embodiments that do.

### SUMMARY

According to a first aspect of the invention, a computer-implemented method for training an artificial intelligence engine for use in estimation of a peripheral artery disease (PAD) status of a patient is provided. Said method comprises: inputting a training data set, the training data set comprising a plurality of samples, wherein each sample comprises a ground truth and motion data comprising at least one motion parameter, determining an estimate ground truth based on the motion data of a sample, comparing the estimate ground truth with the ground truth of the sample, and adjusting the artificial intelligence engine at least partly based on the comparing.

The steps of the above method may be repeated for a plurality of training data sets until the engine achieves a sufficient reliability and/or accuracy. Thereby, the trained engine provides an advantageous tool to determine an estimate ground truth that may be related to a PAD patient's condition in a number- and/or fact-based way exploiting large amounts of available data in an efficient way.

An according exemplary training data set may comprise a plurality of samples, wherein each sample comprises a ground truth and motion data comprising at least one motion parameter. Such training data sets may be provided by collecting motion data from a plurality of PAD patients in a controlled environment and assessing, e.g., their PAD status by means of additional measurements and/or assessments by trained health professionals. Thereby, the PAD patients may provide the motion data and the health professionals the ground truth, e.g., relating to the PAD status according to the health professionals' assessment as described herein.

The training data set may be collected by collecting a plurality of samples by examining a plurality of patients. In this example, each patient may provide the motion data for one sample: E.g., the patient may, in a doctor's appointment be equipped with a medical sensor for a certain time, which may e.g., range from a few minutes to a couple of days or any time therebetween. The motion data collected in this time by the medical sensor provide the motion data that may be comprised in the respective sample, in this example, by counting steps by means of an accelerometer allowing to determine the number of consecutively taken steps without interruption, the duration of walking pauses taken in between and a step frequency. The motion data may be based on data collected by a GPS module. The motion data may comprise a walking speed and a walking distance at a minimum walking speed and within a specific time duration. An attending health professional may assess the patient's PAD status and/or any other possible health indicator that may be useful as a ground truth before, during, and/or after the collection of the motion data for said sample. The ground truth may, e.g., be one label from a predetermined list, a rating on a predetermined numeric scale, etc. This assessment poses the ground truth assigned to the sample. Thereby, each sample comprises motion data, in this example the number of consecutively taken steps without interruption and the duration of walking pauses in between, as well as a ground truth, in this example the PAD status of the patient.

In one example, the ground truth may comprise the PAD status.

This may advantageously provide a trained engine that is configured to provide the exact measure that attending health professionals monitoring PAD patients may be most interested in. This may significantly reduce errors due to human misjudgment, reduce the health professionals' workload and associated costs, and increase the overall quality of care. In fact, it has turned out that a reliable analysis of PAD status may indeed be achieved by training the engine with datasets of patients with motion data and (human-determined) PAD status. Motion of a patient shows reliable correlation with PAD status.

The PAD status may, e.g., comprise a label out of a predetermined group of possible labels, e.g., good/bad and/or any other label as described herein.

According to a further aspect of the invention, a system for estimation of a PAD status of a patient is provided. The system comprises: means for receiving motion data comprising at least one motion parameter and means for determining an estimate PAD status, based at least partly on the motion data.

Such system may provide assistance to attending health professionals for monitoring and/or caring for PAD patients. It may thereby significantly reduce errors due to human misjudgment, reduce the health professionals' workload and associated costs, and increase the overall quality of care.

The means for receiving may, e.g., also be configured to store the received motion data at least partly and/or temporarily until the estimate PAD status is determined.

The system may, e.g., be implemented as a remote (cloud-based) server, a system distributed over multiple user devices that may communicate with one another and/or in one or more medical devices that may acquire and/or provide the motion data.

One or more medical devices may acquire and/or provide the motion data and/or may by wearable, implantable, and/or other medical devices and/or sensors. Suitable devices may, e.g., comprise any of the embodiments described herein. The medical device may, e.g., be a smart watch or a mobile phone utilizing sensors (e.g., accelerometers) for step counting comprised therein.

For example, the at least one motion parameter may comprise at least one of: a walking distance measure, a walking pause measure, a step measure, and a posture measure. Further, the at least one motion parameter may comprise a step frequency, a walking speed, a walking distance at a minimum walking speed and/or within a specific time duration.

These parameters may be particularly suitable to determine the PAD status based thereon, increasing the efficiency, accuracy, and/or reliability of the method and/or system described herein. In particular, pause duration as an expression of perfusion is a very suitable parameter for monitoring. If it decreases, this represents good collateralization, good participation in gait training and a low probability of a necessary re-intervention. However, if the pause duration increases, poor collateralization, poor gait training, or even acute deterioration can be assumed.

The walking distance measure may, e.g., comprise a time period during which the patient was walking (without interruption), a step count relating the steps taken by the patient (without interruption), a distance walked by the patient (without interruption), and/or maximum values, minimum values and/or averages thereof. Generally, measures indicating how far or long the patient could walk without having to take a break may be critical parameters for assessing the PAD patient's PAD status. In some examples, it may comprise the maximum distance walked without interruption. Further, measures indicating the walking speed and step frequency may be critical parameters for assessing the patients's PAD status, as well.

The walking pause measure may, e.g., comprise a pause duration, a pause frequency, a pause frequency variability, and/or maximum values, minimum values and/or averages thereof, it may relate the pause to the distance walked before pausing/taking a break, etc. Generally, measures indicating how often and/or for how long the patient must take a break after walking a certain distance and/or time may be critical parameters for assessing the PAD patient's PAD status. In some examples it may comprise a mean pause duration.

The step measure may, e.g., comprise a step count in a predetermined time window, a maximum number of steps taken without a pause therebetween, a step width, and/or maximum values, minimum values and/or averages thereof. Generally, measures relating to the steps taken by the patient may be critical parameters for assessing the PAD patient's PAD status. In some examples, it may comprise a mean step length.

The posture measure may, e.g., comprise relate to whether the patient is sitting, standing, or walking in a certain time window. Such measures may refine the assessment of the patient's motion, e.g., by being able to differentiate between walking pauses in which the patient was standing and those in which they were sitting or even lying down.

The system may additionally also consider further parameters for determining the PAD status, e.g., cardiovascular parameters which may be indirectly linked to the motion of the patient. Such exemplary further parameters may comprise the oxygen saturation of the patient's blood, their heart rate, and/or further cardiovascular and/or other physiological parameters. Also, (essentially) non-variable parameters may be considered, e.g., the patient's birthday (indicating their age), their height, weight, etc.

Any of the measures described herein may be collected/recorded by sensors and/or medical devices described herein and may at least partly be provided to the means for determining in a processed or unprocessed form.

The estimate PAD status may, e.g., comprise a medical classifier.

This may simplify the use of the system for the involved health professional who may directly see the classifier indicating the PAD status of the patient.

The medical classifier may, e.g., classify the current estimate PAD state by means of a label out of a predetermined group of possible labels, e.g., good/bad, intervention required/no intervention required, good/ok/intermediate/critical/bad/very bad, 1/2/3/4/5, any other group of numerical or non-numerical labels, and/or combinations thereof. The numerical labels may be integers and/or numbers on a continuous spectrum that may classify the PAD status according to a predetermined scale, e.g., from 0 to 10 or 1 to 5, wherein a high number may be an indicator for a good PAD status requiring no intervention and a low number may be an indicator for an alarming PAD status potentially requiring intervention by a health professional, or the other way round

Determining the estimate PAD status may, in some examples, be based at least partly on a comparison of the motion data with predetermined reference data comprising at least one predetermined reference motion parameter.

The predetermined reference motion parameter may pose a useful baseline indicating an ideal value or a lower and/or upper limit defining a range in which a motion parameter may be seen as acceptable or alarming, respectively.

In an example, the comparison may be between an individual motion parameter and a predetermined reference motion parameter.

The comparison may, e.g., comprise calculating a difference between one individual motion parameter and the associated predetermined reference motion parameter. Such difference may, e.g., how far off an ideal value a current motion parameter is. Based thereon, PAD status determination may be facilitated and be more reliable and accurate.

For a more sophisticated assessment, this may also be performed for multiple pairs of motion parameters and predetermined reference motion parameters.

In some examples, the predetermined reference motion parameter(s) may relate to a human determined PAD state of the patient. As long as the comparison leads to deviations between current motion parameters and reference motion parameters (e.g. maximum distance walked without pause, mean pause duration, and/or mean step length), no change of PAD status may be determined. However, if larger deviations occur, a change of PAD status may be detected. In an example, the comparison may be between an aggregate motion score based on a plurality of motion parameters and a reference motion score based on a plurality of predetermined reference motion parameters.

This may provide a more complete assessment considering the influence of multiple motion parameters.

The motion score may, e.g., be determined by the fraction of time spent walking t_{walking}, calculated, e.g., by (t_{walking})/(t_{walking}+t_{pausing}) wherein the sum of the t_{walking} and the time spent pausing t_{pausing} correspond to the full time for which that motion score is determined. For example, in a 10 min time window, a patient might have been walking for t_{walking}=3 min and pausing for t_{pausing}=7 min, which might in this scenario result in a motion score of 30%.

Another example of the motion score may, e.g., translate to the average step width or step length: A reference step width W_{ref}=75 cm may be known for a patient, e.g., adapted to their height (in this example 180 cm). The reference for taller patients may be higher and for smaller patients lower. In an exemplary scenario, the average step width of a patient during a 10 min observation window may be below that reference value W_{avg}=60 cm. The motion score may in this example be calculated by W_{avg}/W_{ref}=80%

In a further example, the motion score may consider both of the above and may be given by the average motion score of the two above examples (30%+80%)/2=55%. Alternatively, the parameters may be weighted differently: E.g., The fraction of time spent walking may be weighted as ¾ contributor to the motion score and the step width only as a ¼ contributor, resulting in a motion score of (¾×30%+¼×80%)=42.5%, using the exemplary values from above.

Other simple and/or more sophisticated algorithms to calculate such motion scores are possible.

In an exemplary embodiment, the means for determining the estimate PAD status may comprise an artificial intelligence engine trained as described herein. Alternatively or in addition, the means for determining the estimate PAD status may be configured to determine the estimate PAD status on basis of a machine learning algorithm.

Thereby the system may run more efficiently, faster, more reliably and/or accurately. Further the artificial intelligence engine may be able to account for more and/or less obvious correlations between the motion data and the PAD status than a simple calculation based on human considerations may utilize.

The system may, e.g., further comprise means for providing information on the estimate PAD status to at least one recipient.

This allows for remote monitoring of the patient which may increase the quality of care, the rate at which the attending health professional may check on the patient, and/or reduce the latency for issue identification.

For example, the information on the estimate PAD status may further comprise information other than the PAD status, preferably a trend of at least one motion parameter and/or the PAD status over time.

Further information may enable the attending health professional, the patient themselves, and/or any other recipient to gain further insights into the PAD status, e.g., its course over time. This may increase the user-friendliness of the system and/or increase treatment success chances due to a more profound understanding of the patient's health development.

The system may, e.g., be configured to provide a suitable visualization of said further information, e.g., in form of charts, graphs, etc.

The system may, e.g., be further configured to provide the information on the estimate PAD status according to a predetermined schedule and/or when at least one motion parameter and/or the PAD state fulfills a predetermined criterion.

This may, e.g., decrease the latency of issue identification, providing the patient with assistance as fast as possible, when needed. This may increase treatment success chances and the efficiency of the system.

The predetermined schedule may, e.g., comprise predetermined times at which the information on the estimate PAD status may be provided to the recipient (e.g., always on Monday, on every weekday from Monday to Friday, etc.) and/or a frequency (e.g., hourly, every six hours, daily, weekly, monthly, and/or any rate therebetween).

The predetermined criterion may, e.g., be directly linked on the PAD status and/or the ground truth. Whenever it indicates an alarming or bad PAD status, the system may automatically send a warning/alarm/notification of the patient and/or the attending health professional.

In an exemplary embodiment, the system may further comprise a user device configured to receive the information on the estimate PAD status and comprising a user-interface for outputting the received information.

This may further improve the user-friendliness of the system, thereby reducing the amount of work required and/or reducing costs associated therewith.

The user device may, e.g., be a computer, a tablet, a mobile phone, and/or any other suitable device, e.g., running an application and/or accessing an (online) portal backend for outputting the received information.

Multiple user devices may, e.g., be comprised in the system allowing multiple users to access the information needed.

The system may, e.g., further comprise a medical sensor for acquiring and providing the motion data, wherein the medical sensor comprises at least one of: an accelerometer, a GPS sensor, a timekeeper, and a position sensor.

These sensors may be particularly suitable to record the motion data described herein providing a solid data basis based on which the PAD status may be determined efficiently and reliably.

E.g., an accelerometer may provide a step count, a GPS sensor may provide a walking distance without interruption, a timekeeper may provide a time of a pause and/or of uninterrupted walking, and/or a position sensor may provide information on the patient's posture. Generally, these sensors may provide any other motion data as described herein.

According to a further aspect, a method for estimation of a PAD status of a patient is provided. The method comprises: receiving motion data comprising at least one motion parameter, determining an estimate PAD status, based at least partly on the motion data, and optionally providing information on the estimate PAD status to at least one recipient.

According to a further aspect of the invention, a computer program for estimation of a PAD status of a patient is provided. The computer program comprises instructions for: receiving motion data comprising at least one motion parameter, determining an estimate PAD status, based at least partly on the motion data, and optionally providing information on the estimate PAD status to at least one recipient.

The method and the computer program therefore provide similar advantages as described herein with reference to systems.

The methods herein may be computer-implemented methods, and a computer program may be provided with instructions according to the steps of the methods. Further, any functionality described herein in reference to a system may be implemented as steps of a method and/or as instructions of a respective computer program, and vice versa.

Computer programs may be stored in any storage device, and the various method steps may be implemented in software or hardware or a combination thereof.

Embodiments of the present disclosure may be realized in any of various forms. For example, in some embodiments, the present invention may be realized as a computer-implemented method, a computer-readable memory medium, or a computer system.

In some embodiments, a non-transitory computer-readable memory medium may be configured so that it stores program instructions and/or data, where the program instructions, if executed by a computer system, cause the computer system to perform a method, e.g., any of the method embodiments described herein, or, any combination of the method embodiments described herein, or, any subset of any of the method embodiments described herein, or, any combination of such subsets.

In some embodiments, a computing device may be configured to include a processor (or a set of processors) and a memory medium, where the memory medium stores program instructions, where the processor is configured to read and execute the program instructions from the memory medium, where the program instructions are executable to implement any of the various method embodiments described herein (or, any combination of the method embodiments described herein, or, any subset of any of the method embodiments described herein, or, any combination of such subsets). The device may be realized in any of various forms.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. In particular, with reference to the appended claims, features from dependent claims may be combined with those of the independent claims and features from respective independent claims may be combined in any appropriate manner and not merely in the specific combinations enumerated in the appended claims.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 shows an exemplary system for estimation of a PAD status of a patient.

FIG. 2 shows an exemplary activity record over time comprising active phases and pause phases.

FIG. 3 shows an exemplary motion score record over time in comparison to a predetermined reference motion score.

FIG. 4 shows a schematic representation of a computer-implemented method for training an artificial intelligence engine for use in estimation of a PAD status of a patient.

### DETAILED DESCRIPTION

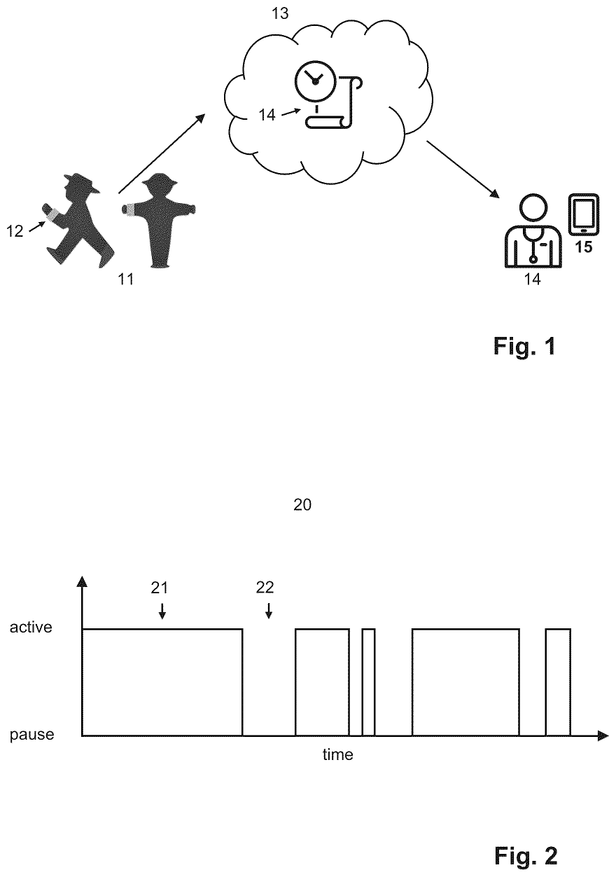

FIG. 1 shows an exemplary system 10 for estimation of a PAD status of a patient 11. In the example of FIG. 1, the patient 11 wears a wearable medical device 12, e.g., attached to their hand wrist. The medical device 12 may, however also comprise an implantable medical device and/or any other medical device. The medical device in FIG. 1 collects certain motion data, e.g., in form of one or more motion parameters, optionally along with further data that may be of interest like the pulse rate and/or the blood oxygen saturation of the patient. For example, these parameters may be measured regularly, e.g., once per second, 10 seconds, 20 seconds, 3 seconds, minute, 10 minutes, etc. and transmitted to the system 13 configured to process these motion data, optionally along with the further data. In the example of FIG. 1, the system 10 is a cloud-based server (system) comprising means for determining 14 an estimate PAD status, based on the motion data, optionally along with the further data. The means for determining 14 then provide an estimate PAD status and information on the estimate PAD status are then provided by to at least one recipient 14, which in this example is a health professional, who may receive said information via a user device 15, e.g., a computer running a suitable application and/or accessing an (online) portal to receive said information. Additionally or alternatively, the system 10 may provide feedback to the patient 11 in such a way that the patient 11 can view the information and optionally receive advice on how to improve their walking distance. In some examples, feedback may be provided by the system 10 and/or the user device 15.

FIG. 2 shows an exemplary activity record 20 over time comprising active phases 21 and pause phases 22. The graph shows multiple active phases 21 in which the patient's status was labelled as “active” and pause phases 22 in which the patient's status was labelled as “pause”. For example, the respective medical device/sensor providing the underlying motion data might have counted for the monitored patient by an accelerometer. Whenever the time difference between two consecutive steps is below a certain first threshold and/or the time period required to take a minimum of e.g., 10, 20, 30 etc. steps does not exceed a certain second threshold, the associated timeslot may be labelled as “active” and as “passive” when the opposite is the case. Such processing may, e.g., be either a preparation for determining the PAD status of the patient based thereon and/or to provide the processed motion data, e.g., in form of the activity record 20 to the recipient for them to get some deeper understanding of the actual motion behavior of the patient. For this, such graph may be provided along with the information on the PAD status, e.g., on a report sheet provided to the recipient.

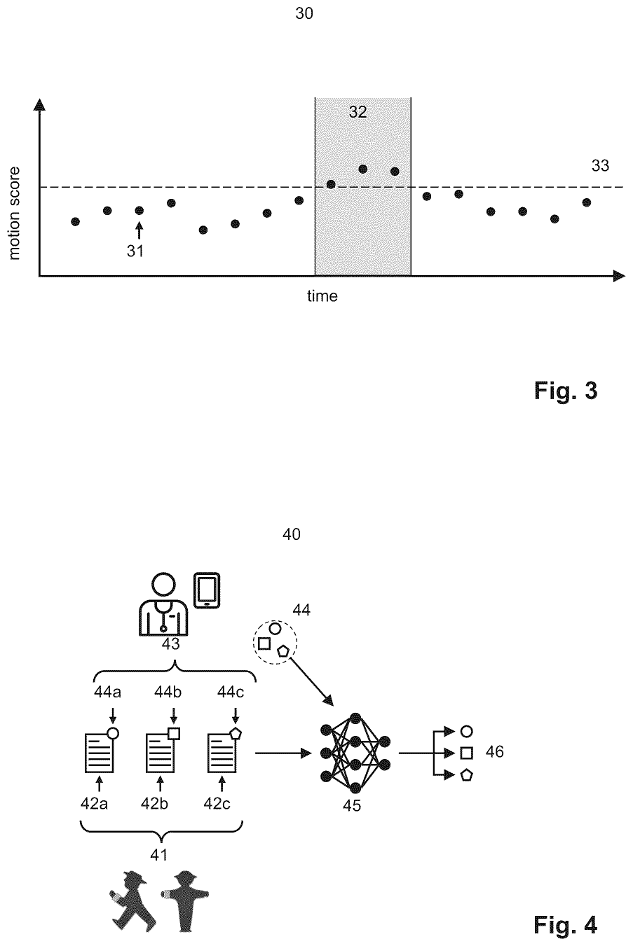

FIG. 3 shows an exemplary motion score record 30 over time in comparison to a predetermined reference motion score 33. In this example, the motion score 31 is determined regularly as shown by the equidistantly distributed motion score data points 31 along the time axis. The motion score 31 may be based on a plurality of motion parameters and the reference motion score 33 may also be based on a plurality of predetermined reference motion parameters. The system processing the motion score may compare the motion score 31 with the reference motion score 33. In this example there is a first time slot 32 in which the motion score 31 exceeds the reference motion score 33. The system may then be triggered to send a notification to a recipient who may, e.g., be asked to provide some medical intervention and/or to check on the patient. Alternatively, such notification may in other examples be sent when the motion score falls below a certain reference motion score 33.

FIG. 4 shows a schematic representation of a computer-implemented method 40 for training an artificial intelligence engine 45 for use in estimation of a PAD status 46 of a patient 41. In the example of FIG. 4, a plurality of motion data 42a, 42b, 42c are gathered by one or more patients equipped with suitable medical devices and/or sensors for collecting at least one motion parameter during an observation interval. Each set of motion data is assigned to one sample, each of which is then also assigned a ground truth 44a, 44b, 44c, e.g., by a health professional 43 who may in this example determine the PAD status within a predetermined range of PAD statuses 44. The engine to be trained may then be provided with the samples and the ground truths 44 to learn how to assign said ground truths to a sample. This process may be done for any plurality of samples until the engine achieves a predetermined accuracy level. The accuracy may, e.g., be determined by using test samples comprising motion data and a ground truth each, which may be unknown to the engine before testing. Upon completion of the training, the engine 45 maybe capable of assigning motion data that are provided to it with the correct ground truth.

Training of such an engine 45 is preferably performed using motion data of PAD patients 41 from a clinical trial with at least 500 patients 41 and over a follow-up period of 6 to 12 months. The input for such training 40 and/or the engine 45 in general may correspond to activity records like the one shown in FIG. 2. A suitable artificial intelligence engine here would be, for example, the Random Forrest algorithm, a Support Vector Machine, a convolutional neuronal network or a multi-layer perceptron.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A computer-implemented method for training an artificial intelligence engine for use in estimation of a peripheral artery disease (PAD) status of a patient, the method comprising:
inputting a training data set, the training data set comprising a plurality of samples, wherein each sample comprises a ground truth and motion data comprising at least one motion parameter;
determining an estimate ground truth based on the motion data of a sample;
comparing the estimate ground truth with the ground truth of the sample; and
adjusting the artificial intelligence engine at least partly based on the comparing.

2. The computer-implemented method of claim 1, wherein the ground truth comprises the PAD status.

3. A system for estimation of a peripheral artery disease (PAD) status of a patient, the system comprising:
means for receiving motion data comprising at least one motion parameter; and
means for determining an estimate PAD status, based at least partly on the motion data.

4. The computer-implemented method of claim 1, wherein the at least one motion parameter comprises at least one of: a walking distance measure, a walking pause measure, a step measure, and a posture measure.

5. The computer-implemented method of claim 1, wherein the estimate PAD status comprises a medical classifier.

6. The system of claim 3, wherein determining an estimate PAD status is based at least partly on a comparison of the motion data with predetermined reference data comprising at least one predetermined reference motion parameter and a predetermined reference PAD status.

7. The system of claim 6, wherein the comparison of the motion data comprises a comparison of at least one individual motion parameter of the motion data.

8. The system of claim 6, wherein the comparison comprises a comparison between an aggregate motion score based on a plurality of motion parameters and a reference motion score based on a plurality of predetermined reference motion parameters.

9. The system of claim 3, wherein the means for determining the estimate PAD status comprises a trained artificial intelligence engine.

10. The system of er claim 3, further comprising means for providing information on the estimate PAD status to at least one recipient.

11. The system of claim 10, further configured to provide the information on the estimate PAD status according to a predetermined schedule and/or when at least one motion parameter and/or the PAD status fulfills a predetermined criterion.

12. The system of claim 10, further comprising a user device configured to receive the information on the estimate PAD status and comprising a user-interface for outputting the received information.

13. The system of claim 3, further comprising a medical sensor for acquiring and providing the motion data, wherein the medical sensor comprises at least one of: an accelerometer, a GPS sensor, a timekeeper, and a position sensor.

14. A method for estimation of a peripheral artery disease (PAD) status of a patient, the method comprising:
receiving motion data comprising at least one motion parameter;
determining an estimate PAD status, based at least partly on the motion data; and optionally providing information on the estimate PAD status to at least one recipient.

15. A computer program for estimation of a peripheral artery disease (PAD) status of a patient, the computer program comprising instructions for:
receiving motion data comprising at least one motion parameter;
determining an estimate PAD status, based at least partly on the motion data; and
optionally providing information on the estimate PAD status to at least one recipient.

16. The system of claim 3, wherein the at least one motion parameter comprises at least one of: a walking distance measure, a walking pause measure, a step measure, and a posture measure.

17. The system of claim 3, wherein the estimate PAD status comprises a medical classifier.
